(19) United States
(12) Patent Application Publication
(10) Pub. No.: US 2025/0057876 A1
(43) Pub. Date: Feb. 20, 2025

(54) METHODS FOR TREATING AND/OR PREVENTING LYMPHANGIOGENESIS-ASSOCIATED DISEASES BY USING GOLD NANOCLUSTERS

(71) Applicants: MacKay Memorial Hospital, Taipei City (TW); Mackay Medical College, New Taipei City (TW); Chung Yuan Christian University, Taoyuan City (TW); GoldRed NanoBiotech CO., LTD., Taoyuan City (TW)

(72) Inventors: Hung-I YEH, Taipei City (TW); Yih-Jer WU, Taipei City (TW); Shih-Wei WANG, Taipei City (TW); Ching-Hu CHUNG, Taipei City (TW); Cheng-Yung LIN, New Taipei City (TW); Wen-Hsiung CHAN, New Taipei City (TW); Kuan-Jung LI, Tainan City (TW); Hong-Shong CHANG, Tao-Yuan City (TW)

(21) Appl. No.: 18/235,717

(22) Filed: Aug. 18, 2023

(51) Int. Cl. A61K 33/242; A61K 9/50; A61K 45/06; A61P 7/10

(52) U.S. Cl. CPC A61K 33/242; A61K 9/5015; A61K 45/06; A61P 7/10

(57) ABSTRACT

Disclosed herein is a method for treating and/or preventing a lymphangiogenesis-associated disease in a subject, including administering to the subject a therapeutically effective amount of a dihydrolipoic acid (DHLA)-coated gold nanocluster about 0.1 to 10 nm in diameter. Also disclosed is a method for promoting lymphangiogenesis in a subject, including administering to the subject an effective amount of said DHLA-coated gold nanocluster.

### BACKGROUND OF THE INVENTION

### 1. Field of the Invention

The present disclosure in general relates to the fields of treating lymphangiogenesis-associated diseases and promoting lymphangiogenesis in a subject. More particularly, the present disclosure relates to methods for treating lymphangiogenesis-associated diseases and promoting lymphangiogenesis by use of a dihydrolipoic acid (DHLA)-coated gold nanocluster.

### 2. Description of Related Art

Lymphangiogenesis-associated diseases refer to a group of disorders characterized by abnormal lymphatic vessel growth, leading to impaired lymphatic drainage and subsequent tissue dysfunction. The pathogenesis of these diseases involves dysregulation of factors involved in lymphatic vessel development and remodeling, such as vascular endothelial growth factors (VEGFs), fibroblast growth factors (FGFs), and angiopoietins. These factors promote lymphatic endothelial cell proliferation, migration, and tube formation. Dysfunctional lymphangiogenesis can result from genetic mutations, inflammation, tumor growth, or chronic conditions such as lymphedema.

Epidemiologically, lymphangiogenesis-associated diseases vary depending on the specific condition. Lymphedema, for example, affects approximately 140-250 million individuals globally, with primary lymphedema often arising from genetic abnormalities and secondary lymphedema resulting from surgery, radiation, or infection. Other conditions like lymphangiomas, lymphangioleiomyomatosis (LAM), and lymphatic malformations have varying prevalence rates and clinical presentations. The treatment approaches currently used for lymphangiogenesis-associated diseases primarily aim to manage symptoms and improve quality of life. Conservative management options may include compression therapy, physical therapy, and skincare measures to reduce tissue swelling and prevent complications in conditions like lymphedema. Surgical interventions, such as lymphaticovenous anastomosis or lymph node transplantation, may be performed in some selected cases. In some instances, pharmacological therapies targeting lymphangiogenesis-associated factors are utilized. For instance, the use of VEGF-C/VEGF-D or some diuretics has shown potential in reducing lymphatic vessel growth in lymphedema. However, these therapeutics are not specifically designed to modulate lymphatic vessel growth and function. Most current treatments focus on managing symptoms and improving quality of life rather than directly targeting the underlying pathogenic mechanisms. Additionally, the heterogeneity and complexity of lymphangiogenesis-associated diseases pose challenges in developing universal treatment strategies.

In view of the foregoing, there exists in the related art a need for an improved method for specifically and effectively treating lymphangiogenesis-associated diseases, thereby effectively addressing the needs of patients with lymphangiogenesis-associated diseases.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

As embodied and broadly described herein, one aspect of the disclosure is directed to a method for treating and/or preventing a lymphangiogenesis-associated disease in a subject. The method comprises administering to the subject an effective amount of a dihydrolipoic acid (DHLA)-coated gold nanocluster having a particle size ranging from 1 to 10 nm; preferably, having a particle size ranging from 1 to 5 nm; more preferably, having a particle size of 2 nm. The DHLA-coated gold nanocluster consists of a gold nanocluster formed by a plurality of gold nanoparticles, and a plurality of DHLAs coated on the gold nanocluster.

According to the embodiments of the present disclosure, the effective amount of the DHLA-coated gold nanocluster is about 0.001-10 mg/kg body weight per day; preferably, about 0.01-1 mg/kg body weight per day. According to one particular working example, the effective amount of the DHLA-coated gold nanocluster is about 0.057 mg/kg body weight per day.

According to the embodiments of the present disclosure, the lymphangiogenesis-associated disease as described herein is lymphedema and systolic heart failure. Thus, in some advanced examples, the present method may further comprise administering an additional medicament in combination with the DHLA-coated gold nanocluster to treat the above indicated diseases. The additional medicament may be administered to the subject prior to, in conjunction with, or subsequent to the administration of the DHLA-coated gold nanocluster.

For example, when the lymphangiogenesis-associated disease is lymphedema, the additional medicament may be at least one agent selected from the group consisting of VEGF-C, VEGF-D, acetazolamide, furosemide, azosemide, bumetanide, etacrynic acid, etozolin, indacrinone, muzolimine, ozolinone, piretanide, tienilic acid, torasemide, altizide, bendroflumethiazide, butizide, chlorothiazide, cyclopenthiazide, cyclothiazide, epitizide, hydrochlorothiazide, hydroflumethiazide, mebutizide, methyclothiazide, polythiazide, trichiormethiazide, chlortalidone, clofenamide, clopamide, clorexolone, fenquizoneindapamide, mefruside, meticrane, metolazone, quinethazone, xipamide, amiloride, benzamil, triamterene, spironolactone, canrenone, eplerenone, potassium canrenoate, finerenone, conivaptan, mozavaptan, satavaptan, and tolvaptan.

Alternatively, when the lymphangiogenesis-associated disease is systolic heart failure, the additional medicament may be any one agent selected from the group consisting of carvedilol, metoprolol, bisoprolol, losartan, valsartan, candesartan, sacubitril, lisinopril, enalapril, ramipril, spironolactone, eplerenone, digoxin, hydralazine, and isosorbide dinitrate, ivabradine, atorvastatin, simvastatin, aspirin, clopidogrel, warfarin, apixaban, rivaroxaban, metolazone, nebivolol, and milrinone.

Preferably, the subject is a human.

Another aspect of the present disclosure pertains to a method for promoting lymphangiogenesis in a subject. The method comprises administering to the subject an effective amount of a DHLA-coated gold nanocluster. The DHLA-coated gold nanocluster consists of a gold nanocluster formed by a plurality of gold nanoparticles, and a plurality of DHLAs coated on the gold nanocluster.

According to some embodiments of the present disclosure, the DHLA-coated gold nanocluster is about 1 to 10 nm in diameter; preferably, about 1 to 5 nm in diameter; more preferably, about 2 nm in diameter.

According to the embodiments of the present disclosure, the effective amount of the DHLA-coated gold nanocluster is about 0.001-10 mg/kg body weight per day; preferably, about 0.01-1 mg/kg body weight per day. According to one particular working example, the effective amount of the DHLA-coated gold nanocluster is about 0.057 mg/kg body weight per day.

Preferably, the subject is a human.

Many of the attendant features and advantages of the present disclosure will becomes better understood with reference to the following detailed description considered in connection with the accompanying drawings.

### BRIEF DESCRIPTION OF THE DRAWINGS

These and other features, aspects and advantages of the present invention will become better understood with reference to the following description, appended claims and the accompanying drawings, where:

FIGS. 1A-1B are the diagrams depicting the effect of DHLA-coated gold nanoclusters (indicated as fluorescent gold nanoclusters (FANCs) in figures) on cell growth (FIG. 1A) and migration (or wound healing; FIG. 1B) in human lymphatic endothelial cells (LECs). Data are expressed as mean±S.E.M, of at least five independent experiments. *, P<0.05, compared to the control group.

FIG. 2 is the diagram depicting the effect of FANCs on tube formation in human LECs. Data are expressed as mean±S.E.M. of at least five independent experiments. *, P<0.05, compared to the control group.

FIG. 3 is the diagram depicting the expression of a lymphatic endothelial specific marker, lymphatic vessel endothelial hyaluronan receptor-1 (LYVE-1), in isoproterenol (ISO)- and/or FANC-treated mice. Data are presented as mean±S.E.M. Control, N=4; ISO, N=3; FANC+ISO, N=6. *, P<0.05, compared with the ISO group.

FIGS. 4A-4B are the diagrams depicting the results of echocardiography in ISO-, FANC-, and/or MAZ-51-treated mice, in which MAZ-51 is (3E)-3-[[4-(dimethylamino)naphthalen-1-yl]methylidene]-JH-inden-2-one. FIG. 4A shows the results of fractional shortening (FS), and FIG. 48 shows the results of tissue doppler imaging (TDI-s′). Data are presented as mean±S.E.M. Control, N=3; ISO, N=3; FANC+150, N=3; FANC+ISO+MAZ-51, N=4. *, P<0.05, compared with the control group; †, P<0.05, compared with the ISO group; ‡, P<0.05, compared with the ISO+FANC group.

FIG. 5 is the diagram depicting the in vivo lymphangiogenesis in FANC-treated zebrafishes, showing the percentage of embryos with more than 7 somites containing intersegmental lymphatic vessels (ISLV) in the 5th to 12th somites. Each experiment was obtained from 20 larvae. Data are presented as mean±S.E.M. from at least three independent experiments. *, P<0.05, compared with the control group.

### DETAILED DESCRIPTION OF THE INVENTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### I. Definition

For convenience, certain terms employed in the specification, examples and appended claims are collected here. Unless otherwise defined herein, scientific and technical terminologies employed in the present disclosure shall have the meanings that are commonly understood and used by one of ordinary skill in the art. Also, unless otherwise required by context, it will be understood that singular terms shall include plural forms of the same and plural terms shall include the singular. Specifically, as used herein and in the claims, the singular forms “a,” “an,” and “the” include the plural reference unless the context clearly dictates otherwise. Also, as used herein and in the claims, the terms “at least one” and “one or more” have the same meaning and include one, two, three, or more. The practice of the present invention will employ, unless otherwise indicated, conventional techniques of biochemistry, general biology, animal experimentation, and the like, which are within the skill of the art. Such techniques are explained fully in the literature.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about” Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

The terms “treatment” and “treating” as used herein may refer to an intervention performed with the intention of preventing the development or altering the pathology of a lymphangiogenesis-associated disease. The concept of treatment or treating is used in the broadest sense, and specifically includes the prevention (prophylaxis), moderation, reduction, curing, and palliation of lymphangiogenesis-associated diseases of any stage. Accordingly, “treatment” refers to both therapeutic treatment and prophylactic or preventative measures, or even refers to palliative measures, wherein the object is to prevent or slow down (lessen) or ameliorate a lyrnphangiogenesis-associated disease such as lymphedema and systolic heart failure. Those in need of treatment include those already with the disease as well as those prone to have the disease or those in whom the disease is to be prevented. The disease may result from any cause, including idiopathic or cardiotropic causes, or secondary to these causes or other conditions, such as surgery, radiation, or infection.

The term “subject” or “patient” refers to an animal including the human species that is treatable with the method of the present disclosure. The term “subject” or “patient” intended to refer to both the male and female gender unless one gender is specifically indicated. Accordingly, the term “subject” or “patient” comprises any mammal which may benefit from administration of the DHLA-coated gold nanocluster. Examples of a “subject” or “patient” include, but are not limited to, a human, a rat, a mouse, a guinea pig, a monkey, a pig, a goat, a cow, a horse, a dog, a cat, a bird, and a fowl. According to a preferred embodiment, the subject is a human.

The term “administered,” “administering,” or “administration” are used interchangeably herein to refer either directly administering the present DHLA-coated gold nanocluster, or a pharmaceutical composition comprising the DHLA-coated gold nanocluster.

The term “an effective amount” as used herein refers to an amount of the gold nanoclusters effective, at dosages, and for periods of time necessary, to achieve the desired result with respect to the treatment of lymphangiogenesis-associated disease or promoting lymphangiogenesis in a subject. For therapeutic purposes, the effective amount is also one in which any toxic or detrimental effects of the component are outweighed by the therapeutically beneficial effects. The specific effective or sufficient amount will vary with such factors as the particular condition being treated, the physical condition of the patient (e.g., the patient's body mass, age, or gender), the type of mammal or animal being treated, the duration of the treatment, the nature of concurrent therapy (if any), and the specific formulations employed and the structure of the compounds or its derivatives. Effective amount may be expressed, for example, in grams, milligrams or micrograms or as milligrams per kilogram of body weight (mg/kg). Alternatively, the effective amount can be expressed in the concentration of the active component (e.g., the present DHLA-coated gold nanocluster), such as molar concentration, mass concentration, volume concentration, molality, mole fraction, mass fraction and mixing ratio. Persons having ordinary skills could calculate the human equivalent dose (HED) for the medicament (such as the present DHLA-coated gold nanocluster) based on the doses determined from animal models. For example, one may follow the guidance for industry published by US Food and Drug Administration (FDA) entitled “Estimating the Maximum Safe Starting Dose in Initial Clinical Trials for Therapeutics in Adult Healthy Volunteers” in estimating a maximum safe dosage for use in human subjects.

The term a “lymphangiogenesis-associated disease” as used herein refers to a disease, condition, or disorder that is originated from or exacerbated by compromised lymphangiogenesis in a subject; a lymphangiogenesis-associated disease, for example, may be lymphedema or systolic heart failure.

The term “pharmaceutical composition” refers to a preparation which is in such form as to permit the biological activity of an active ingredient contained therein to be effective, and which contains no additional components which are unacceptably toxic to a subject to which the composition would be administered. In general, the amount of active compounds (i.e., the present DHLA-coated gold nanocluster) is present in the pharmaceutical composition at a level of about 0.01% to 99% by weight; preferably, at a level of at least 0.1% by weight; more preferably, at a level of at least 1% by weight; even more preferably, at a level of at least 5% by weight; yet even more preferably, at a level of at least 10% by weight; still yet even more preferably, at a level of at least 25% by weight, based on the total weight of the pharmaceutical composition. For the clinical use of the present invention, the present pharmaceutical composition is formulated into formulations suitable for the intended route of administration.

The phrase “pharmaceutically acceptable excipient” as used herein means a pharmaceutically acceptable material, composition or vehicle, such as a liquid or solid filler, diluent, carrier, solvent or encapsulating material, involved in carrying or transporting the subject agents from one organ, or portion of the body, to another organ, or portion of the body. Each excipient must be “acceptable” in the sense of being compatible with the other ingredients of the formulation. The pharmaceutical formulation contains the present DHLA-coated gold nanocluster in combination with one or more pharmaceutically acceptable ingredients. The excipient can be in the form of a solid, semi-solid or liquid diluent, cream or a capsule. These pharmaceutical preparations are a further object of the invention.

### II. Description of the Invention

The present disclosure is based, at least in part, on the discovery that the DHLA-coated gold nanoclusters are found to enhance lymphangiogenesis features both in vitro (such as increasing cell growth, migration, and tube formation) and in vivo (such as improving cardiac functions in cardiac injured mice and promoting lymphangiogenesis in zebrafishes), suggesting DHLA-coated gold nanoclusters are potential candidates for the development of medicaments for the treatment and/or prophylaxis of lymphangiogenesis-associated diseases.

The present DHLA-coated gold nanocluster structurally consists of a gold nanocluster formed by a plurality of gold nanoparticles, and a plurality of DHLA-coated on the gold nanocluster. A skilled practitioner is familiar with the DHLA-coated gold nanoclusters utilized in the present disclosure, as well as their production method (Lin et al., ACS Nono 2009, 3 (2), pp 395-401); hence further elaboration on the details for their preparation is not necessary in this regard. The DHLA-coated gold nanoclusters have a fluorescent emission at 650 nm under an excitation wavelength of about 420 nm, hence the emitted fluorescence is in the range of red to near infrared spectrum. Each of the DHLA-coated gold nanocluster has a particle size ranging from 0.1 to 20 nm, preferably ranging from 1 to 10 nm, more preferably ranging from 1 to 5 nm, and even more preferably at 2 nm. The dimension discussed above related to the gold nanocluster of the present disclosure is in dry state, however, it is of advantage if the gold nanocluster used in the present disclosure is water-soluble or at least dispersible in aqueous medium and/or water; the hydrodynamic size of the gold nanocluster of the present disclosure can be significantly larger than that in the dry state due to the coupling of surrounding solvent molecule, such as water. In one specific embodiment example, the gold nanocluster has a hydrodynamic size corresponds to 1 to 30 kDa polyethylene glycol (PEG) molecules (Lin et al, ACS Nano 2009, 3 (2), pp 395-401).

According to one aspect of the present disclosure, the DHLA-coated gold nanoclusters are used to prepare a composition for treating and/or preventing a lymphangiogenesis-associated disease or promoting lymphangiogenesis in a subject; such composition may be utilized in the form of a pharmaceutical composition or medicament, or a health functional food (including a nutritional supplement) according to its purposes. The composition comprises the DHLA-coated gold nanoclusters in an effective amount, along with an optional pharmaceutically acceptable excipient.

Methods for preparing the pharmaceutical composition are well known in the art of pharmacology. For the application of the present invention, the DHLA-coated gold nanoclusters of the present disclosure may be manufactured into desired formulations, such as tablets, sugar-coated tablets, pills, granules, aerosols, syrups, emulsions, suspensions, solutions, ointments, creams or gels or any kind, in particular by using inert, essentially nontoxic, pharmaceutically suitable excipients.

According to some embodiments of the present disclosure, the composition is used as the pharmaceuticals, in such case, the pharmaceutical composition may further comprise suitable carriers, excipients, and diluents commonly used in the manufacture of pharmaceutical compositions. The pharmaceutical composition may be formulated and used in the form of oral dosage forms such as powders, granules, tablets, capsules, suspensions, emulsions, syrups, aerosols, external preparations, suppositories; and sterile injectable solutions according to a conventional method. Carriers, excipients, and diluents that may be included in the pharmaceutical composition comprise lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate, and mineral oil. In the case of formulation, it is prepared using excipients or diluents such as fillers, extenders, binders, wetting agents, disintegrating agents, and surfactants. Solid preparations for oral administration include tablets, pills, powders, granules, capsules, etc. These solid preparations include at least one excipient in the preparation, for example, starch, calcium carbonate, sucrose, lactose, gelatin, and the like. In addition, lubricants such as magnesium stearate or talc are used in addition to simple excipients. Liquid preparations for oral use include suspending agents, solutions, emulsions, syrups, etc. In addition to water and liquid paraffin, which are commonly used as simple diluents, various excipients, such as wetting agents, sweeteners, fragrances, and preservatives, may be included in the preparation. Formulations for parenteral administration include sterile aqueous solutions, non-aqueous solvents, suspensions, emulsions, lyophilized preparations, and suppositories. Non-aqueous solvents and suspensions may include propylene glycol, polyethylene glycol, vegetable oils such as olive oil, and injectable esters such as ethyl oleate. As a base for suppositories, witepsol, macrogol, Tween-61, cacao butter, laurin butter, and glycerogelatin may be used.

According to some embodiments of the present disclosure, the composition is utilized as the health functional food, which can be any kinds of liquid and solid/semi-solid materials that are used for nourishing humans and animals, for benefiting treatment of a lymphangiogenesis-associated disease and/or ameliorating or alleviating the symptoms associated with the lymphangiogenesis-associated disease. Ingredients commonly used in the health functional food may be included in addition to the active ingredient, for example, citric acid, oligosaccharide, taurine, fruit concentrate, etc. may be included when the composition is manufactured as a health functional beverage. The health functional food may be a food product (e.g., tea-based beverages, juice, soft drinks, coffee, milk, jelly, cookies, cereals, chocolates, snack bars, herbal extracts, dairy products (e.g., ice cream, and yogurt)), a food/dietary supplement, or a nutraceutical formulation.

The DHLA-coated gold nanoclusters provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

In another aspect of the present disclosure, the present disclosure provides methods for treating lymphangiogenesis-associated diseases and promoting lymphangiogenesis in a subject. The method comprises administering to the subject an effective amount of a DHLA-coated gold nanocluster, or a composition comprising the same, as described herein.

In certain embodiments, the DHLA-coated gold nanoclusters described herein are provided in an effective amount in the pharmaceutical composition. In certain embodiments, the effective amount is a therapeutically effective amount (e.g., amount effective for treating a lymphangiogenesis-associated disease). In certain embodiments, the effective amount is a prophylactically effective amount (e.g., amount effective for preventing a lymphangiogenesis-associated disease in a subject in need thereof).

The exact amount of DHLA-coated gold nanoclusters required to achieve an effective amount will vary from subject to subject, depending, for example, on species, age, and general condition of a subject, severity of the side effects or disorder, mode of administration, and the like. An effective amount may be included in a single dose (e.g, single oral dose) or multiple doses (e.g., multiple oral doses). In certain embodiments, when multiple doses are administered to a subject, any two doses of the multiple doses include different or substantially the same amounts of DHLA-coated gold nanoclusters described herein. In certain embodiments, when multiple doses are administered to a subject, the frequency of administering the multiple doses to the subject is three doses a day, two doses a day, one dose a day, one dose every other day, one dose every third day, one dose every week, one dose every other week, one dose monthly or one dose every other month. In certain embodiments, the frequency of administering the multiple doses to the subject is one dose per day. In certain embodiments, the frequency of administering the multiple doses to the subject is two doses per day. According to some examples of the present disclosure, the subject is a mouse. To elicit a therapeutic effect on mice, the present DHLA-coated gold nanoclusters is administered to the subject in the amount of about 0.01-150 mg/kg body weight per day; preferably, about 0.1-15 mg/kg body weight per day; more preferably, about 0.1-1.5 mg/kg body weight per day. According to one working example, the present DHLA-coated gold nanoclusters are administered to the subject in the amount of 0.57 mg/kg body weight per day.

A skilled artisan may readily determine the human equivalent dose (HED) of the present DHLA-coated gold nanoclusters, based on the doses determined from animal studies provided in working examples of this application. Accordingly, the amount of the present DHLA-coated gold nanoclusters suitable for use in a human subject may be in the range of 0.001-10 mg/Kg body weight per day; for example, 0.001. 0.002, 0.003, 0.004, 0.005, 0.006, 0.007, 0.008, 0.009, 0.01, 0.011, 0.012, 0.013, 0.014, 0.015, 0.016, 0.017, 0.018, 0.019, 0.02, 0.021, 0.022, 0.023, 0.024, 0.025, 0.026, 0.027, 0.028, 0.029, 0.03, 0.031, 0.032, 0.033, 0.034, 0.035, 0.036, 0.037, 0.038, 0.039, 0.04, 0.041, 0.042, 0.043, 0.044, 0.045, 0.046, 0.047, 0.048, 0.049, 0.05, 0.051, 0.052, 0.053, 0.054, 0.055, 0.056, 0.057, 0.058, 0.059, 0.06, 0.061, 0.062, 0.063, 0.064, 0.065, 0.066, 0.067, 0.068, 0.069, 0.07, 0.071, 0.072, 0.073, 0.074, 0.075, 0.076, 0.077, 0.078, 0.079, 0.08, 0.081, 0.082, 0.083, 0.084, 0.085, 0.086, 0.087, 0.088, 0.089, 0.09, 0.091, 0.092, 0.093, 0.094, 0.095, 0.096, 0.097, 0.098, 0.099, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, or 10 mg/kg body weight per day. Preferably, the amount of the present DHLA-coated gold nanoclusters for treating a human subject is about 0.01-1 mg/kg body weight per day. More preferably, the amount of the present DHLA-coated gold nanoclusters for treating a human subject is about 0.01-0.1 mg/kg body weight per day. According to one working example, the amount of the present DHLA-coated gold nanoclusters for treating a human subject is about 0.046 mg/kg body weight per day. According to one working example, the amount of the present DHLA-coated gold nanoclusters for treating a human subject is about 0.057 mg/kg body weight per day.

Dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

The DHLA-coated gold nanocluster and compositions comprising such provided herein can be administered by any suitable route, including enteral (e.g., oral), parenteral, intravenous, intramuscular, intra-arterial, intramedullary, intrathecal, subcutaneous, intraventricular, transdermal, interdermal, subcutaneous, intradermal, rectal, intravaginal, intraperitoneal, topical (as by powders, ointments, creams, and/or drops). Specifically, contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration).

As would be appreciated, the present method can be applied to the subject, alone or in combination with additional therapies that have some beneficial effects on the treatment of the lymphangiogenesis-associated disease. Depending on the intended purpose, the present method can be applied to the subject before, during, or after the administration of the additional therapies. The specific type of additional treatment drug will depend on the particular condition being treated (i.e, lymphedema or systolic heart failure).

In the case when the lymphangiogenesis-associated disease is lymphedema, the additional medicament may be at least one agent of VEGF-C, VEGF-D, acetazolamide, furosemide, azosemide, bumetanide, etacrynic acid, etozolin, indacrinone, muzolimine, ozolinone, piretanide, tienilic acid, torasemide, altizide, bendroflumethiazide, butizide, chlorothiazide, cyclopenthiazide, cyclothiazide, epitizide, hydrochlorothiazide, hydroflumethiazide, mebutizide, methyclothiazide, polythiazide, trichlormethiazide, chlortalidone, clofenamide, clopamide, clorexolone, fenquizoneindapamide, mefruside, meticrane, metolazone, quinethazone, xipamide, amiloride, benzamil, triamterene, spironolactone, canrenone, eplerenone, potassium canrenoate, finerenone, conivaptan, mozavaptan, satavaptan, or toivaptan.

In the case when the lymphangiogenesis-associated disease is systolic heart failure, the additional medicament may be any one agent of carvedilol, metoprolol, bisoprolol, losartan, valsartan, candesartan, sacubitril, lisinopril, enalapril, ramipril, spironolactone, epierenone, digoxin, hydralazine, and isosorbide dinitrate, ivabradine, atorvastatin, simvastatin, aspirin, clopidogrel, warfarin, apixaban, rivaroxaban, metolazone, nebivolol, or milrinone.

In some embodiments, the subject is mammal, preferably is human.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

### EXAMPLE

### Materials and Methods

### 1. Preparation of DHLA-Coated Gold Nanoclusters (FANCs)

Fluorescent DHLA-coated gold nanoclusters used in this study were prepared based on precursor-induced gold (Au) nanoparticle (NP) etching (Lin et at, ACS Nano, 2009, 3 (2), pp 395-401). Briefly, 6-nm gold nanoparticles stabilized with didodecyldimethylammonium bromide (DDAB) were firstly synthesized in toluene via adding 0.8 ml gold precursors (AuCl₃, 7.5 mg/ml prepared in 100 mmol/L DDAB solution) into the reduction agents containing 1 ml of fresh-prepared tetrabutylammonium borohydride (TBAB) (100 mmol/L in DDAB solution) as well as 0.675 ml decanoic acid (100 mmol/L in toluene). Additional gold precursors were dropwise added until the color changed to transparent yellow, resulting in non-plasmonic gold nanoclusters which can be further brought to aqueous phase upon ligand exchange with reduced ca-lipoic acid. α-lipoic acid (0.206 g, Sigma-Aldrich, St. Louis, Missouri, USA) was reduced into dihydrogenliopic acid (DHLA) by adding 5 ml TBAB (50 mmol/L) in DDAB solution. DHLA-coated gold nanoclusters were formed by mixing equal amount of gold precursors with DHLA and being subjected to additional UV light (302 nm) annealing for 30 minutes, resulting in the stable fluorescence signal in living cells. For the sake of brevity, the DHLA-coated gold nanoclusters are abbreviated as “FANCs.” After removing the supernatants by centrifugation, FANCs were further purified by following methanol/chloroform washing steps and redisposed in borate buffer (pH 9) for overnight incubation under 55° C., and collected and buffer-exchanged by centrifugal filter between 30-100 kDa (EMD Millipore).

### 2. Cell Culture

Human lymphatic endothelial cells (LECs) were obtained from commercially available sources (Lonza; Walkersville, MD). The LECs were grown in endothelial cell growth medium (EGM-2MV medium) comprising EBM-2 basal medium plus endothelial cell growth supplement (Lonza). The cells were seeded onto 1% gelatin-coated plasticware and cultured at 37° C. with 5% CO₂ for subsequent treatment.

### 3. Cell Growth Assay

The LECs were seeded onto 96-well plates in at the density of 5×10³ cells per well. After a 24-hour incubation, the culture medium was removed, and the cells were treated with EGM-2MV medium in the absence or presence of FANCs for an additional 24 hours. Subsequently, the cells were fixed with 50% trichloroacetic acid (TCA) to terminate the reaction, and treated with 0.4% sulforhodamine B (SRB) (Sigma; St. Louis, MO, USA) in 1% acetic acid. After a 15-min incubation, the plates containing the cells were washed and treated with 10 mM Tris buffer. The absorbance of the plate at 515 nm was measured using an enzyme-linked immunosorbent assay (ELISA) reader.

### 4. Cell Migration Assay

The LECs were seeded into a 6-well plate and allowed to grow until they reached 90-100% confluence. A wound was created on the cells by scraping a gap using 1 mm-sized scratcher tip (SPL Life Sciences, Pocheon, Korea), and the cells were washed with Hanks' balanced salt solution (HBSS) twice. The cells were incubated in fresh growth medium with or without FNACs at the indicated concentrations. Images were captured by a microscope at 0, 16, and 24 hours after treatment and were subsequently subjected to analyses. The migration area was calculated using Image J with MRI Wound Healing Tool.

### 5. Capillary Tube Formation Assay

Matrigel (BD Biosciences; Bedford, MA) was dissolved overnight at 4° C., and 96-well plates were coated with 50 μl Matrigel in each well, followed by incubation at 37° C. for 30 minutes. The LECs were seeded at a density of 2×10⁴/200 μl in EGM-2MV medium per well with the indicated concentrations of FANCs. After a 24-hour incubation at 37° C., the tube formation of the LECs was evaluated by microscopy, and photographs were taken of each well. The number of tube branches and total tube length were calculated using the MacBiophotonics Image J software.

### 6. Isoproterenol (ISO)-Induced Heart Failure Model

Male 7-weeks-old C57BL/6 mice were purchased from BioLasco Inc. (Yilan, Taiwan), The mice were treated with FANCs (20 μM; about 0.57 mg/kg body weight) via an abdominal implant using osmotic pump system on Day 0. Then, the mice were subcutaneously treated with ISO (150 mg/kg) on Day 14. After 14 days from ISO injection, echocardiography was performed on Day 28 to measure the cardiac function of the mice. The animals were then sacrificed, and their hearts were harvested for histological and pathological examination.

### 7. Immunohistochemistry (IHC) Staining

The mouse left ventricle tissue sections were deparaffinized with xylene and rehydrated by adding ethanol. Endogenous peroxidase activity was blocked with 3% hydrogen peroxide in methanol for 10 minutes. Heat-induced antigen retrieval was carried out for all sections in 0.01 M sodium citrate buffer, pH 6 at 95° C. for 25 minutes. Antibodies against mouse LYVE-1 were applied at a dilution of 1:200 and incubated at 4° C. overnight. The antibody-binding signal was detected using the NovoLink Polymer Detection System (Leica Microsystems) and visualized with the diaminobenzidine reaction. The sections were counterstained with hematoxylin and eosin (H&E). The quantification of LYVE-1 expression was analyzed using MacBiophotonics Image-J software.

### 8. Echocardiography

The experimental echocardiography for the mice was conducted after the mice were anesthetized with isoflurane (Abbott Laboratories Ltd., England; delivered to achieve a concentration of 1-2% in a combination of oxygen and nitrous oxide mixed at a ratio of 50:50 with air). All the mice underwent cardiac structure and function assessment using a 10-MHz sector array transducer connected to a Vivid i echo system (GE Vingmed Ultrasound, Horten, Norway), The mice were positioned in left lateral position with ultrasound gel placed on the anterior chest wall. The depth of M-mode linear scan was set at 1.0-1.5 cm with the highest frame rate achievable. Parameters including tissue Doppler used to determine contractile velocity (TDI-S′), deformation-based circumferential systolic strain measure, and quantitative indices of tissue structure integrated backscatter intensity from dynamic myocardial motions, were used to assess systolic myocardial performance using ECG-gated off-line analysis on the EchoPac Workstation.

### 9. In Vivo Lymphangiogenesis Model

A DNA construct used to establish a transgenic zebrafish strain Tg(Iyve1:TRFP) was generated as follows: A 6.2 kb promoter sequence of the lymphatic vessel endothelial hyaluronan receptor 1b (Iyve1b) gene was linked to the red fluorescent protein (TRFP) to drive its expression, so as to allow the marking of developing lymphatic vessel cells in the transgenic zebrafish. The transgenic zebrafish line Tg(Iyve1:TRFP) were established using the aforementioned DNA construct accordingly. The red fluorescence signal were detected in the majority of the growing lymphatic vessel cells in the head and trunk regions of zebrafish embryos at 144 hours post-fertilization (hpf). FANCs at a concentration of 5 nM were injected into the zebrafish line Tg(Iyve1:TRFP) 72 hpf embryo, and the lymphatic vessel growth in the trunk region was observed at 144 hpf. Finally, the percentage of embryos with more than 7 somites containing intersegmental lymphatic vessels (ISLV) in the 5th to 12th somites was determined to evaluate in vivo lymphangiogenic function.

### 10. Data Analysis

Experiment results expressed as mean±S.E.M. were analyzed using one-way ANOVA or Student's t-test. Consider the data are statistically significant when P value<0.05.

### Example 1 In Vitro Effects of FANCs on Lymphangiogenesis

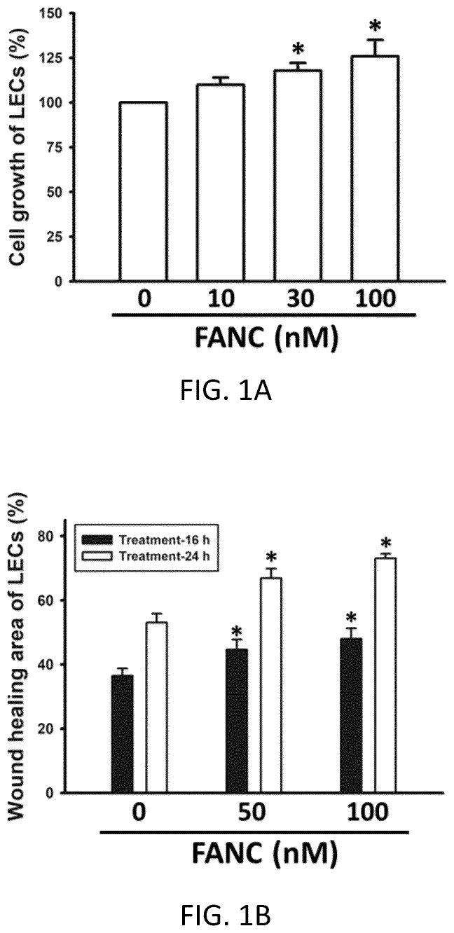

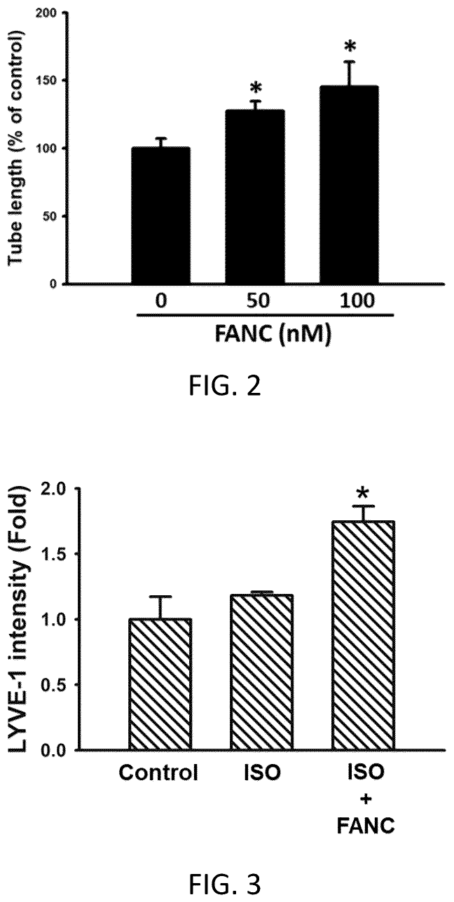

The effects of FANCs on lymphangiogenesis, including promoting cell growth, enhancing cell migration, and promoting the formation of capillary-like structure in lymphatic endothelial cells (LECs), were investigated in this example. Results are provided in FIGS. 1 and 2. Specifically, it was found that FANCs increased the cell growth of LECs in a concentration-dependent manner (FIG. 1A). As to the effects of FANCs on migration and tube formation abilities, which are crucial for endothelial cells during lymphangiogenesis, it was found that FANCs significantly increased the wound healing area of LECs in a concentration- and time-dependent manner (FIG. 11), and significantly induced the formation of capillary-like structures in LECs in a concentration-dependent manner (FIG. 2).

Taken together, the results collectively suggested that FANCs possessed the ability to promote in vitro lymphangiogenesis in human LECs.

### Example 2 In Vivo Effects of FANCs on Lymphangiogenesis and Cardiac Function in Isoproterenol (ISO)-Induced Cardiac Injury Mice

The in vivo effects of FANCs on lymphangiogenesis were investigated via using an isoproterenol (ISO)-induced cardiac injury mouse model, which is a typical pathogenesis of post-myocardial infarction (MI) and heart failure model. Immunohistochemical analysis was first performed to examine LYVE-1, a key specific marker for lymphatic vessels, in order to validate the role of lymphangiogenesis in the therapeutic effect mediated by FANCs. It is noted that FANCs significantly increased the expression of LYVE-1 compared to the cardiac tissues derived from the ISO-treated group, as shown in FIG. 3. The results substantiated that FANCs can enhance lymphangiogenesis in cardiac injured mice.

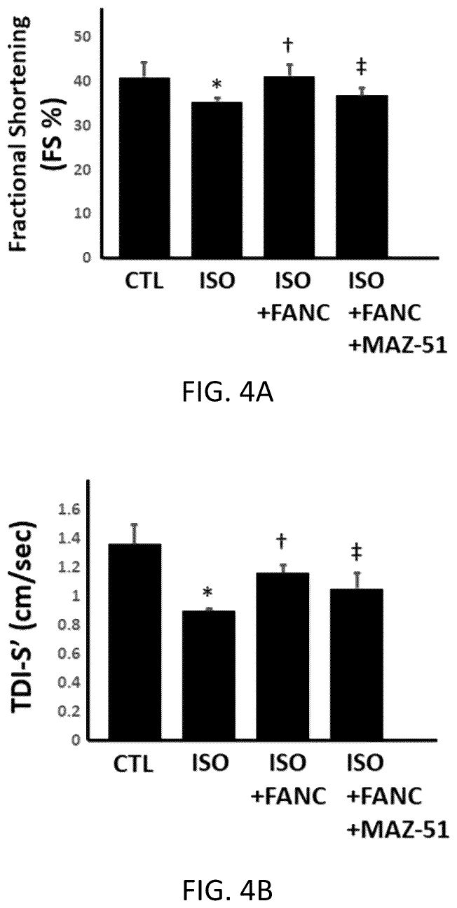

As FANCs improved lymphangiogenesis in ISO-induced cardiac injured mice, thus, the cardiac function of these mice were further investigated. As depicted in FIGS. 4A-4B, the treatment with FANCs improved fractional shortening (FS) and tissue Doppler imaging (TDI-s′) in ISO-treated mice. FS serves as an indicator for ventricular systolic function, whereas TDI-s′ serves as an indicator for myocardial systolic velocity. The results clearly demonstrated that, with lymphangiogenesis in cardiac injured tissue improved after treatment with FANCs, an improved cardiac function ensued. In addition, the FANCs improved cardiac function was abolished by co-treatment of MAZ-51, a lymphangiogenesis inhibitor, further confirmed that the FANCs enhanced cardiac function was attributed to its ability in promoting lymphangiogenesis. Taken together, these results substantiate that FANCs have the ability to promote lymphangiogenesis in hearts, which in turn improves cardiac function in cardiac injured mice.

### Example 3 In Vivo Effects of FANCs on Lymphangiogenesis in Zebrafishes

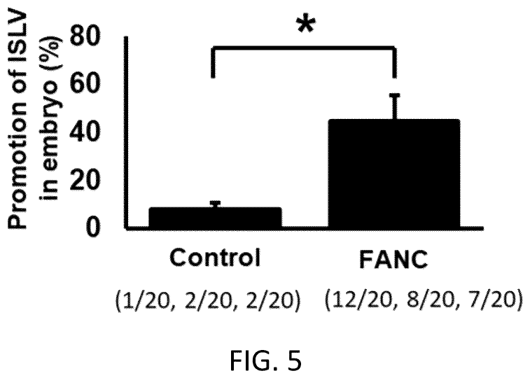

In this example, the in vivo effect of FANCs on lymphangiogenesis was explored in zebrafishes. To this end, a transgenic zebrafish strain Tg(Iyve1:TRFP) was created, in which a 6.2 kb promoter of lymphatic vessel endothelial hyaluronan receptor 1b (Iyve1b) gene was used to drive the expression of red fluorescent protein (TRFP), so as to enable the visualization of developing lymphatic vessel cells, particularly in allowing the observation of the head and trunk regions of zebrafish embryos at 144 hours post-fertilization (hpf). FANCs were administered intraperitoneally into Tg(Iyve1:TRFP) zebrafish embryos at 72 hpf, and the growth of lymphatic vessel in the trunk region was observed at 144 hpf. The percentage of embryos with intersegmental lymphatic vessel (ISLV) growth in more than 7 out of the 8 somites between the 5th to 12th somite was calculated. The untreated control group exhibited a percentage of 8.3±2.4%, whereas the FANC-treated group showed a percentage of 45.0±10.8% (FIG. 5), Based on these findings, once again, the promotion of lymphatic vessel growth by FANCs was demonstrated in the zebrafish model.

In conclusion, data in working examples demonstrated that the DHLA-coated gold nanoclusters can facilitate lymphangiogenesis in vitro and in vivo. Accordingly, the administration of DHLA-coated gold nanoclusters to individuals in need may potentially ameliorate conditions resulting from abnormal lymphangiogenesis, such as lymphedema and systolic heart failure. The present method, which comprises the step of administering the DHLA-coated gold nanoclusters and/or a pharmaceutical composition comprising the same, may provide a potential means to effectively treat or prevent a subject from suffering from lymphangiogenesis-associated diseases.

It will be understood that the above description of embodiments is given by way of example only and that various modifications may be made by those with ordinary skill in the art. The above specification, examples and data provide a complete description of the structure and use of exemplary embodiments of the invention, Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. A method for treating and/or preventing a lymphangiogenesis-associated disease in a subject comprising administering to the subject an effective amount of a dihydrolipoic acid (DHLA)-coated gold nanocluster having a particle size ranging from 1 to 10 nm, wherein the DHLA-coated gold nanocluster consists of a gold nanocluster formed by a plurality of gold nanoparticles, and a plurality of DHLAs coated on the gold nanocluster.

2. The method of claim 1, wherein the DHLA-coated gold nanocluster has a particle size ranging from 1 to 5 nm.

3. The method of claim 2, wherein the DHLA-coated gold nanocluster has a particle size of 2 nm.

4. The method of claim 1, wherein the gold nanocluster is administered in the amount of about 0.001-10 mg/kg body weight per day.

5. The method of claim 4, wherein the gold nanocluster is administered in the amount of about 0.01-1 mg/kg body weight per day.

6. The method of claim 1, wherein the lymphangiogenesis-associated disease is lymphedema and systolic heart failure.

7. The method of claim 6, further comprising administering to the subject at least one agent selected from the group consisting of VEGF-C, VEGF-D, acetazolamide, furosemide, azosemide, bumetanide, etacrynic acid, etozolin, indacrinone, muzolimine, ozolinone, piretanide, tienilic acid, torasemide, altizide, bendroflumethiazide, butizide, chlorothiazide, cyclopenthiazide, cyclothiazide, epitizide, hydrochlorothiazide, hydroflumethiazide, mebutizide, methyclothiazide, polythiazide, trichlormethiazide, chlortalidone, clofenamide, clopamide, clorexolone, fenquizoneindapamide, mefruside, meticrane, metolazone, quinethazone, xipamide, amiloride, benzamil, triamterene, spironolactone, canrenone, eplerenone, potassium canrenoate, finerenone, conivaptan, mozavaptan, satavaptan, and toivaptan, when the lymphangiogenesis-associated disease is lymphedema.

8. The method of claim 6, further comprising administering to the subject an agent selected from the group consisting of carvedilol, metoprolol, bisoprolol, losartan, valsartan, candesartan, sacubitril, lisinopril, enalapril, ramipril, spironolactone, eplerenone, digoxin, hydralazine, and isosorbide dinitrate, ivabradine, atorvastatin, simvastatin, aspirin, clopidogrel, warfarin, apixaban, rivaroxaban, metolazone, nebivolol, and milrinone, when the lymphangiogenesis-associated disease is systolic heart failure.

9. The method of claim 1, wherein the subject is a human.

10. A method for promoting lymphangiogenesis in a subject comprising administering to the subject an effective amount of a DHLA-coated gold nanocluster having a particle size ranging from 1 to 10 nm, wherein the DHLA-coated gold nanocluster consists of a gold nanocluster formed by a plurality of gold nanoparticles, and a plurality of DHLAs coated on the gold nanocluster.

11. The method of claim 10, wherein the DHLA-coated gold nanocluster has a particle size ranging from 1 to 5 nm.

12. The method of claim 11, wherein the DHLA-coated gold nanocluster has a particle size of 2 nm.

13. The method of claim 10, wherein the gold nanocluster is administered in the amount of about 0.001-10 mg/kg body weight per day.

14. The method of claim 13, wherein the gold nanocluster is administered in the amount of about 0.01-1 mg/kg body weight per day.

15. The method of claim 10, wherein the subject is a human.
